(19) United States
(12) Patent Application Publication
(10) Pub. No.: US 2011/0281329 A1
(43) Pub. Date: Nov. 17, 2011

(54) PROCESS AND SYSTEM FOR THE INDUSTRIAL SCALE PURIFICATION OF BACTERIOPHAGES INTENDED FOR BACTERIOPHAGE THERAPY

(75) Inventors: Hansjorg Lenherr, Zola Predosa-Bologna (IT); Reinhard Bartsch, Zola Predosa-Bologna (IT)

(73) Assignee: GIMA S.P.A., Zola Predosa-Bologna (IT)

(21) Appl. No.: 12/679,071

(22) PCT Filed: Oct. 2, 2007
(86) PCT No.: PCT/IT07/00684

§ 371 Date: Mar. 19, 2010

(51) Int. Cl. C12N 7/02; C12M 1/12

(52) U.S. Cl. 435/239; 435/294.1

(57) ABSTRACT

The present invention is directed towards bacteriophage therapy, the treatment of infectious diseases, caused by pathogenic bacteria, using specific bacteriophages. The purity of bacteriophage preparations has been a major obstacle for their therapeutic use in the past. Current purification methods are only suitable for low-scale production, as they are work and cost intensive. Disclosed is a method and machinery to purify crude bacteriophage preparations in large scale and fully automated, resulting in highly pure (toxin free) and highly concentrated bacteriophage preparations, suitable for agricultural and clinical applications.

The present invention deals with a process and a system for the industrial scale purification of bacteriophages intended for bacteriophage therapy.

As an adaptation to the selective environment they encounter, many pathogenic bacteria have become resistant to a wide range of antibiotics [9]. Alternative forms of treating such resistant bacteria are thus in high demand. Bacteriophage therapy is one of the alternatives considered today. Worldwide, the interest in bacteriophage therapy is on the rise, after it has been dormant in the Western World, for almost 60 years [6]. Past applications of bacteriophage therapy have been hampered by the inability to purify bacteriophage preparations in order to remove exo- and endotoxins, as well as to preserve the biological activity of the bacteriophages [8]. Current methods to produce and purify bacteriophages are derived from laboratory methods and are not suitable for large scale preparations. The present invention relates to the large scale production and purification of bacteriophage preparations in order to combat infectious diseases, especially, but not exclusively, when the bacteria causing these diseases are resistant to antibiotics. It describes a method to produce, on an industrial scale, bacteriophage compositions that are highly concentrated and free of any toxic remnants (bacterial debris, bacterial endo- and exotoxins), that are the byproduct of the production process.

The use of bacteriophages to fight bacterial infections has already been proposed by d'Hérelle, the co-discoverer of the bacteriophages [2]. However, while some of the early applications proved to be successful, others failed, in retrospect mainly due to the lack of knowledge about the interactions between bacteriophages and their host bacteria [11]. With the advent of the antibiotic area any attempts to establish bacteriophages as antimicrobial agents were abandoned in Western medicine. On the other hand, bacteriophages were widely used in the former Soviet Union and Poland, but unfortunately, most of the Eastern studies demonstrating the efficacy of bacteriophage therapy do not meet the standards of our current medical systems. However, bacteriophages served as major study tools in the field of molecular biology in both East and West, and thus have been intensively investigated in many laboratories over the last eighty years [1]. The knowledge gained from this basic research should now prove useful to prepare improved bacteriophage preparations that will fulfil the strict criteria required for both animal and human applications [7, 11].

In the present invention, bacteriophages are amplified in large-scale fermenters or alternatively on semi-solid medium (see US patent application 2007/001001), producing hundreds of litres of solution enriched in bacteriophages, but contaminated with bacterial debris, toxins and components of the bacterial growth medium. Such a solution is then passed through a series of filters in order to separate the bacteriophages from these contaminating substances. In the process the bacterial growth medium containing the bacteriophages is substituted by a solution suitable for packaging and long term storage of the bacteriophages.

The present invention can be used to purify any bacteriophage in large scale, resulting in preparations that can be used directly as disinfecting agents or medical products for both animal and human use.

Bacteriophages are omnipresent in the environment. Methods to isolate and enrich new bacteriophages with desired host ranges are thus known to those skilled in the art [4, 5, 10]. Theoretically it is possible to isolate bacteriophages that grow on any bacterial pathogen known. However, not all bacteriophages found in nature are also suitable for practical applications. A precise genetic characterization of the isolated phages will be essential to select those that destroy their host bacteria with high efficiency and do not transfer any unwanted traits [8]. To identify those bacteriophages most suited for a specific task is a skill in itself (see for example WO 2004/004495). Also, various methods to improve natural isolates or modify their properties exist, as described in U.S. Pat. Nos. 5,811,093 or 7,087,226.

Usually bacteriophages are grown in liquid cultures using fermenters of various sizes [3]. Alternatively bacteriophages can also be grown on semi-solid medium as disclosed in US patent application 2007/0010001. However, either of these two methods creates equally large volumes of liquid phage-containing solutions that need to be purified for further use. The current standard laboratory procedure, as outlined below, follows a work intensive, multi-step protocol and thus is only suitable for smaller volumes of liquid.
1. The bacteriophage-containing solution is cleared by centrifugation.
2. Polyethylene glycol is added to the liquid in order to precipitate the bacteriophages. Polyethylene glycol is a non-specific, reversible crosslinker that precipitates all the proteins present in the solution. The precipitation process takes approximately 12-18 hours at 4 degrees centigrade.
3. Precipitation of the bacteriophages by centrifugation.
4. Resuspension of the precipitated bacteriophages in the minimal amount of a suitable liquid. During this step the bacteriophages are usually concentrated about 50 fold.
5. This solution is then carefully layered onto a CsCl₂-density gradient and processed by centrifugation in an ultracentrifuge. In this step the bacteriophages are separated from other proteins and impurities according to their specific density. This process takes approximately 24 hours.
6. Isolation of the bacteriophages using a syringe with a sharp-tipped needle. This process requires a steady hand and some experimental skill.
7. Removal of the CsCl₂-solution by dialysis (12 to 24 hours) against a buffer of choice.

While this procedure results in highly pure bacteriophage preparations, it appears obvious that it cannot be applied to purify bacteriophages on an industrial scale. Not only is the process time consuming, but it requires an inordinate amount of manual work and is not cost effective either.

In Georgia and the former Soviet Union, bacteriophages were not purified to this extent [3]. Usually bacteriophage solutions only were filter-sterilized and then directly packaged for applications. However, such a procedure does not remove a large number of bacterial proteins, among them toxins and almost all the components of the bacterial growth medium, making such solutions unsuitable for present applications.

The above and other objects and advantages of the invention, as will appear from the following description, are obtained with a process and a system as respectively disclosed in the independent claims. Preferred embodiments and non-trivial variations of the present invention are the subject matter of the dependent claims.

The present invention will be better described by some preferred embodiments thereof, provided as a non-limiting example, with reference to the enclosed drawings, in which:

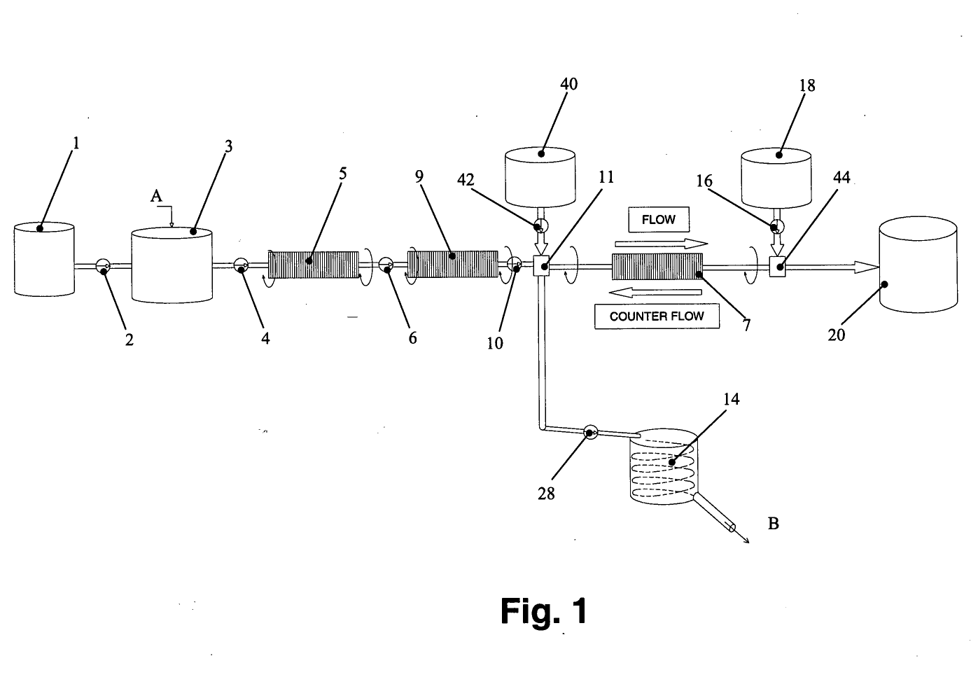

FIG. 1 is a schematic view of a preferred embodiment of the system to which the process of the present invention can be applied;

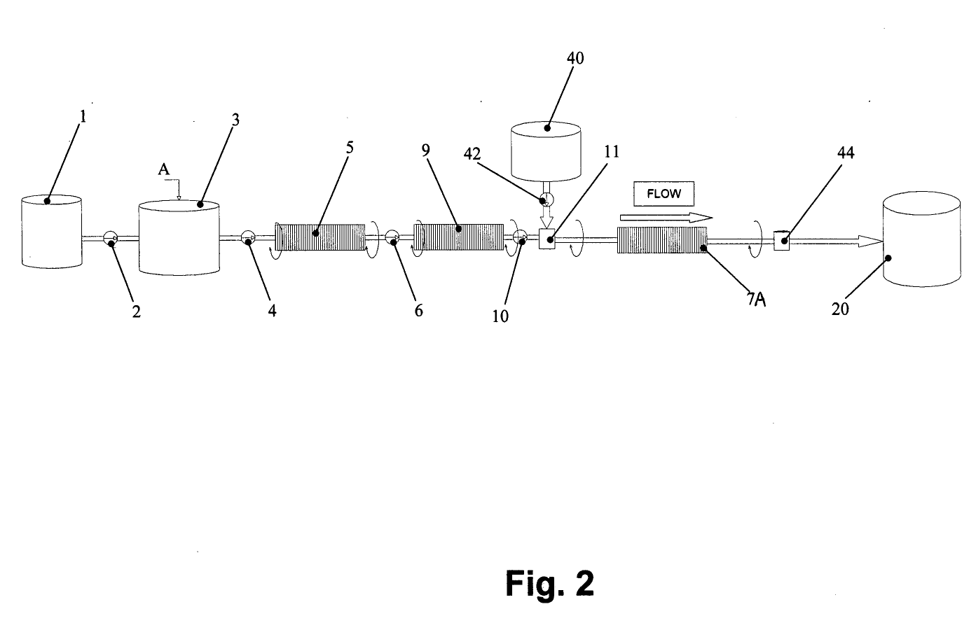

FIG. 2 is a schematic view of the first part of another preferred embodiment of the system to which the process of the present invention can be applied; and

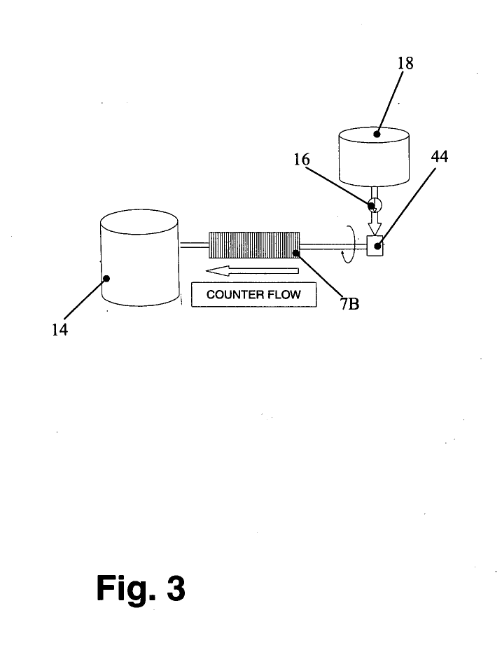

FIG. 3 is a schematic view of the second part of another preferred embodiment of the system to which the process of the present invention can be applied.

With reference to the Figures, preferred embodiments of the process and system of the present invention are shown and described. It will be immediately obvious that numerous variations and modifications (for example related to shape, sizes and parts with equivalent functionality) can be made to what has been described, without departing from the scope of the invention as appears from the enclosed claims.

The present invention relates to a novel purification method that is based on a series of filtration steps that require no manual input, is thus suitable for large scale, industrial production and results in bacteriophage compositions that are highly pure and essentially free of any residual toxic remnants. These purified bacteriophage preparations can then be packaged in any form suitable for their appropriate agricultural or clinical applications.

On average bacteriophages have a diametre of 50 to 100 nanometres and are 100 to 300 nanometres long. They are composed of proteins that form a protective shell around the genetic material. This protective shell contains delicate structures in the nanometre range, which, when broken, result in the loss of the biological activity of the bacteriophages. Special care has thus to be taken in order not to damage the bacteriophages during the purification process.

The procedure for producing and purifying can be divided into the following steps.

### Step 1. Amplification of Bacteriophages.

Procedures to produce large numbers of bacteriophages are known to those skilled in the art. Normally host bacteria are allowed to grown in a fermenter 1 in order to reach high cell densities. Another fermenter 3 containing such bacteria supplied by a pump unit 2 is then seeded with high or low numbers of bacteriophages (arrow A in FIG. 1) depending on whether a single step amplification or a multistep amplification should take place. Alternatively bacteriophages can also be grown on semi-solid medium as disclosed in US patent application 2007/0010001. Depending on the bacteriophage to be amplified and the host bacterium in use, either of these methods results in equally large volumes of bacteriophage preparations containing 10⁹ to 10¹² bacteriophages per millilitre. In this invention a setup using two fermenters 1, 3 is proposed, which will be immediately advantageous when more than one bacteriophage has to be purified.

### Step 2. Prepurification.

The goal of this first purification step is to remove contaminants that are larger than the bacteriophages from the bacteriophage preparations. After the amplification step that results in the destruction of most of the host bacteria, the bacteriophage preparations are contaminated with intact bacterial cells, bacterial cell wall fragments, bacterial lipids in membrane vesicles of various sizes, bacterial proteins, among them exo- and endotoxins and various components of the bacterial growth medium (salts, sugars, proteins). This solution is rich in solids and highly viscous. The state of the art purification scheme using polyethylene glycol precipitation and density centrifugation (as described above) is work intensive and not suitable for large volumes. Traditional filtration techniques, like the standard sterile filtration using filters with 200 nm pores, are very inefficient, as they quickly clog when faced, with such highly viscous solutions. As a result large aggregates are forming, which trap the bacteriophages and drastically reduce the yield. In this invention, the use of a crossflow rotation filtration system with two filtering units 5, 9 is described, but a single filtering unit 9 (or more than two filtering units) could instead be used, however, with an increased risk of clumping. The bacteriophage preparation is pumped, with a pump unit 4, directly from the fermenter 3 to the filtration unit 5, as shown in FIG. 1, and using low pressures (1 bar) the pump unit 4 passes the solution through a Teflon filter 5, that has a pore size of ˜1000 nanometres and rotates with the speed of 400 rotations per minute. Small particles, among them the bacteriophages, pass through the membrane as filtrate, while intact bacterial cells, large bacterial cell wall fragments and bacterial lipids in large membrane vesicles stay behind in the retenate. The low pressure and the rotation of the filter 5 discs prevent large particles from blocking the filter pores. The shearing forces occurring during such a filtration step are relatively low and do not affect the viability of the bacteriophages.

The filtrate of the first filtration unit 5 then serves directly as the feed for a second filtration unit 9, being fed by a pump unit 6. This second filtration unit 9 has an equivalent setup, but the pore size of the Teflon filter is reduced to ˜200 nanometres. Again the bacteriophages, bacterial proteins, sugars and salts pass this second filter, while residual bacterial debris are held back in the retenate. The shearing forces occurring during this second filtration step are already considerable, but experiments showed that more than 95% of the bacteriophages manage to pass such a filter intact. Other filter systems could be used, but with increased risk of clumping and thus lower yield.

### Step 3. Purification.

The filtrate from the prepurification is used as feed for a third filtration unit 7 through a pump unit 10. The goal of this step is to clear the bacteriophage preparations from contaminants that are smaller than the bacteriophages. This is achieved by passing the solution over a filter 7 with a pore size of ˜60 nanometres. The bacteriophages are kept in solution in the retenate, while small proteins, among them the toxins, sugars and salts pass the filter and can be discarded and collected in a collecting tank 20. In a regular filtration, little concern is given to the fate of the retenate, as the filtrate is in general the product. Here, however, the bacteriophages are retained in the retenate and special care has to be taken in order to prevent them from clumping or being inactivated by shearing forces. State of the art crossflow filtration systems are not suitable for such a process, as the shearing forces created by a flow, over a filter with a pore size of ˜60 nanometres are in a range sufficient to inactivate bacteriophages. Thus, in this invention, a milder form of filtration is described. Using very low pressure (0.2 bar), the bacteriophage preparations are passed through a slowly rotating ceramic filter (200 rotations per min). The low pressure and the rotation of the filter 7 prevent the bacteriophages from coagulating on the filter surface.

Once a batch of the phage preparation is processed, the feeding pump 10 is turned off and a second pump 42 passes a cleaning solution, stored in a vessel 40, through a group of valves 11 and into the filter 7. This washing step with cleaning solution guarantees that no components of the original bacterial growth medium are present in the final product, while still keeping the bacteriophages in solution in the retenate.

### Step 4. Elution.

After the cleaning, a third pump 16 at the back of the filtration unit 7 is activated and pumps a storage solution, contained in a vessel 18, through a group of valves 44, in the opposite direction (“COUNTERFLOW” direction in FIG. 1), using an impulse pressure. Otherwise the same conditions as during step 3 are used. In this storage solution, the highly pure bacteriophages are thus eluted from the rotation filter 7. The impulse pressure is used to remove potential deposits from the pores and the surface of the filter. Using appropriate amounts of storage solution to elute, provides an easy and safe mean to adjust the final concentration of the bacteriophages and thus guarantees a standardized high quality end product.

The purified bacteriophage solutions exiting the filter 7 pass through the group of valves 11 and are pumped, through a fourth pump unit 28 (such pump unit 28 is optional, namely its function can be performed, for example, by the pump unit 42), into an intermediate storage container 14. From the intermediate storage container 14, the bacteriophage solutions can then be packaged directly, either in liquid form or dried after lyophilization, following a normal working flow (arrow B in FIG. 1.).

The ideal filtration process is described in FIG. 1, with a fully automated workflow, resulting in highly purified bacteriophages, with essentially no losses during the process. However, depending on the production conditions, it might be necessary to separate the “back flow” filtration step from the rest of the process, in order to gain more flexibility. Thus, FIGS. 2 and 3 show a second embodiment of the system of the invention. The same designation references are kept for the parts with similar or identical functionality.

The main difference between the system of FIG. 1 and the system of FIGS. 2 and 3 is that there are two identical, but separate rotary filters 7A, 7B, one of which performs the operation designated with “FLOW” in FIG. 2 and the other one of which performes the operation designated with “COUNTERFLOW” in FIG. 3; while the rotary filter 7A in FIG. 2 is connected to the collecting tank 20 for wastes, the rotary filter 7B in FIG. 3 is connected to the vessel 18 with the storage solution for counterflow, and to the intermediate storage tank 14 for the final solution of purified bacteriophages.

As another possible embodiment of the inventive system, derived again from FIGS. 2 and 3, the rotary filter 7 could be again only one, as in the first embodiment, shown in FIG. 1, but it could be operated separately in two different steps, one similar to FIG. 2 where the solution moves along the “FLOW” direction, and another one similar to FIG. 3, where the solution moves along the “COUNTERFLOW” direction, obviously performing the two separate steps described above for these two directions and operating steps.

Especially when entire groups of fermenters have to be processed simultaneously, it might be necessary to quickly regenerate the filter system. By removing the rotary filter unit 7A, 7B, rinsing and inserting a fresh filter unit, the entire filtration system as outlined in FIG. 2 will be available to process the content of a second fermenter without delay. The removed filter unit 7A, 7B, containing the purified bacteriophages, can then be processed in a separate elution unit, as outlined in FIG. 3. The filtration system as outlined in FIG. 1 will be designed in order to allow a choice between the two embodiments of the invention.

The systems shown in the Figures are obviously only examples of the different systems that can be used to practice the process of the invention. For example, the filters 5 and 9 could be crossflow rotary filters as shown in the Figures, or could be traditional filters adapted to perform the same filtering functions. Moreover, such filters could be one, two or more, according to the desired filtration task.

Moreover, the intermediate storage tank 14 could be the one shown in FIG. 1, where the solution of purified bacteriophages is subjected to a spiral rotation or swirling adapted to obtain an homogeneous distribution of bacteriophages. Alternatively, the intermediate storage tank 14 could be the traditional one shown in FIG. 3 (that can obviously be used also in the system of FIG. 1) where the final solution of purified bacteriophages is not subjected to rotation or swirling.

### LIST OF REFERENCES

1. Calendar, R. 2006. The Bacteriophages. Oxford University Press, NY.
2. d'Hérelle, F. 1926. The Bacteriophage and its Behavior. Williams and Wilkins, Baltimore, Md.
3. Häusler, T. 2003. Gesund durch Viren. Ein Ausweg aus der Antibiotika-Krise. Piper Verlag GmbH, München.
4. Hoff, J. C. and C. H. Drake. 1962. Simplified method for isolation and purification of bacteriophages. J. Bacteriol. 83:924-92b.
5. Hook, A. E., D. Beard, A. R. Taylor, D. G. Sharp, and J. W. Beard. 1946. Isolation and characterization of the T2 bacteriophage of Escherichia coli. J. Biol. Chem. 165:241-258.
6. Kutter, E. 1997. Phage therapy: Bacteriophage as antibiotics. http://www.evergreen.edu/user/T4/PhageTherapy/Phagethea.html.
7. Lehnherr, H. 2006. Bacteriophage P1, p. 351-364. In R. Calendar (ed.), The Bacteriophages. Oxford University Press, NY.
8. Merrill, C. R., D. Scholl, and S. Adhya. 2006. Phage Therapy, p. 725-742. In R. Calendar (ed.), The Bacteriophages. Oxford University Press, NY.
9. Neu, H. C. 1992. The crisis in antibiotic resistance. Science 257:1064-1073.
10. Putnam, F. W., L. M. Kozloff, and J. C. Neil. 1949. Biochemical studies of virus reproduction. I. Purification and properties of Escherichia coli bacteriophage T6. J. Biol. Chem. 179:303-323.
11. Summers, W. C. 2001. Bacteriophage therapy. Annu. Rev. Microbiol. 55:437-451.

1. A process for an industrial scale purification of bacteriophages intended for bacteriophage therapy, comprising the steps of:
growing host bacteria of the bacteriophages;
mixing bacteria and bacteriophages in order to allow the bacteriophages to grow;
first filtering a preparation of mixed bacteria and bacteriophages, letting the bacteriophages pass through;
first rotation-filtering, wherein the bacteriophages remain in solution in a retenate;
second rotation-filtering with counterflow with respect to the first rotation-filtering, wherein the bacteriophages are subjected to elution; and
storing the solution of purified bacteriophages.

2. The process of claim 1, wherein the process further comprises, after the first filtering step, a second filtering step of the preparation of mixed bacteria and bacteriophages.

3. The process of claim 1, wherein the first filtering step and/or the second filtering step are crossflow rotary filtering steps, the first filtering step being performed by using pores whose size is equal to 1000 nanometres, the second filtering step being performed by using pores whose size is equal to 200 nanometres.

4. The process of claim 1, wherein, in the storing step, the solution of bacteriophages is subjected to spiral rotation or swirling adapted to obtain an homogeneous distribution of bacteriophages.

5. The process of claim 1, wherein the process further comprises the step of packaging the solution of bacteriophages in liquid form.

6. The process of claim 1, wherein the process further comprises the step of packaging the solution of bacteriophages in dry form after lyophilization.

7. A system for an industrial scale purification of bacteriophages intended for bacteriophage therapy, comprising:
a first fermenter, preferably of a high cell density chemiostatic type, adapted to perform a continuous growth of host bacteria;
a second fermenter connected to the first fermenter and adapted to perform a mixing of bacteria and bacteriophages in order to allow the bacteriophages to grow;
a filtering unit connected to the second fermenter and adapted to filter a preparation of bacteria and bacteriophages letting the bacteriophages pass through; and
a rotary filtering unit connected to the filtering unit adapted to perform a first rotation-filtering, wherein the bacteriophages remain in solution in a retenate, and a second rotation-filtering with counterflow with respect to the first rotation filtering, wherein the bacteriophages are subjected to elution.

8. The system of claim 7, wherein the system further comprises:
a first pump unit connected to the rotary filtering unit and adapted to pump an elution solution through the rotary filtering unit, retaining the bacteriophages in the retenate;
an intermediate storage container for the solution of purified bacteriophages; and
a second pump unit connected to the rotary filtering unit and adapted to pump a cleaning solution through the rotary filtering unit into the intermediate storage container.

9. The system of claim 7, wherein the system further comprises:
a first pump unit connected to the rotary filtering unit and adapted to pump an elution solution of the bacteriophages in the retenate;
an intermediate storage container for the solution of purified bacteriophages;
a second pump unit connected to the rotary filtering unit and to the intermediate container and adapted to pump a cleaning solution into the rotary filtering unit;
a third pump unit connected to the rotary filtering unit and adapted to pump into the solution of bacteriophages, a counterflow solution, and then to pump both solutions into the rotary filtering unit for the counterflow in an opposite direction; and
optionally, a fourth pump unit connected to the rotary filtering unit and adapted to pump the final solution of clean bacteriophages into the intermediate storage container.

10. The system of claim 7, wherein, in the intermediate storage container, the obtained solution is subjected to rotation or swirling to obtain a homogeneous and purified distribution of bacteriophages.

11. The system of claim 7, wherein the filtering unit has pores whose size is equal to 1000 nanometres, and the system comprises a further filtering unit containing pores whose size is equal to 200 nanometres.

12. The system of claim 11, wherein the filtering unit and the further filtering unit are of a rotary type.

13. The system of claim 12, wherein the rotary filtering unit is divided into two identical, separate rotary filtering units, a first one of the separate rotary filtering units being connected to the filtering unit and being adapted to perform a first rotation-filtering, wherein the bacteriophages remain in solution in the retenate, a second one of the separate rotary filtering units being connected to the first rotary filtering unit and being adapted to perform a second rotation-filtering with counterflow with respect to the first rotary filtering unit, the bacteriophages being subjected to elution and the obtained solution being subjected to rotation or swirling to obtain a homogeneous and purified distribution of bacteriophages.

14. The system of claim 12, wherein the rotary filtering unit is adapted to firstly perform a first rotation-filtering, wherein the bacteriophages remain in solution in the retenate, and afterwards is adapted to separately perform a second rotation-filtering with counterflow with respect to the first rotation-filtering, wherein the bacteriophages are subjected to elution and the obtained solution is subjected to rotation or swirling to obtain a homogeneous and purified distribution of bacteriophages.

15. The system of claim 12, wherein the rotary filtering unit is adapted to be detached from one of the systems and the detached rotary filtering unit is adapted to be inserted in another one of the systems.
